(19) United States
(12) Patent Application Publication
(10) Pub. No.: US 2019/0069958 A1
(43) Pub. Date: Mar. 7, 2019

(54) CORONARY VEIN GUIDING SYSTEM

(71) Applicant: Beijing Chest Hospital, Capital Medical University, Beijing (CN)

(72) Inventors: Jian ZHANG, Beijing (CN); Liang ZHANG, Beijing (CN)

(21) Appl. No.: 15/948,673

(22) Filed: Apr. 9, 2018

(30) Foreign Application Priority Data
Sep. 4, 2017 (CN) 201710785387.X

(51) Int. Cl. A61B 34/20; A61B 8/12; A61B 8/00; A61B 17/00

(52) U.S. Cl. CPC A61B 34/20; A61B 8/12; A61B 8/445; A61M 2025/0166; A61B 8/4461; A61B 2017/00292; A61B 2034/2063; A61B 17/00234

(57) ABSTRACT

A coronary vein guiding system, comprising a coronary vein guiding catheter, and a flexible inner tube sheathed into the coronary vein guiding catheter sequentially comprising a coaxial section, an anti-bending section and a torsion control section; an end portion of the coaxial section being provided with a flexible head end; the coaxial section being connected with the anti-bending section at obtuse angle of 136°-140°; the anti-bending section being connected with the torsion control section at obtuse angle of 138°-142°; the coaxial section and the torsion control section being situated on a same side of the anti-bending section, and the three sections being located on a same plane; the flexible inner tube being 18-22 cm longer than the coronary vein guiding catheter; a head end of the flexible inner tube being in same direction as the coaxial section, a tail end of the flexible inner tube being provided with a screw joint.

### CROSS-REFERENCE TO RELATED APPLICATIONS

This application claims priority to Chinese Patent Application No. 201710785387.X, filed on Sep. 4, 2017 with the State Intellectual Property Office (SIPO) of the People's Republic of China and entitled “Coronary Vein Guiding System”, the contents of which are herein incorporated by reference in their entirety.

### TECHNICAL FIELD

The present disclosure relates to the field of medical instruments, and particularly to a coronary vein guiding system.

### BACKGROUND ART

Since a new era of interventional cardiology was opened up by doctor Gruentzig carrying out the first percutaneous transluminal coronary angioplasty (PTCA) in the world in 1977, during the development process of more than thirty years, percutaneous coronary intervention (PCI) has experienced the stages of simple balloon dilation and bare metal stent (BMS) implantation and has developed into the modern drug-eluting stent age. More and more complicated coronary artery diseases have been listed as the indications. As one of the indications of percutaneous coronary intervention, chronic total occlusion (CTO) has a low operation success rate, relatively complex operations, relatively high complications and poor long-term prognosis due to its special pathological and anatomical features, compared with non-lesion operations, and is still the last difficulty that has not been overcome in the field of cardiovascular intervention. Compared with the non-CTO lesions, the CTO lesions are not ideal in both the success rate and the therapeutic effect.

The CTO lesion is a lesion that no antegrade flow is seen when coronary artery is radiographed, with the occlusion having lasted for more than three months, which is referred to as true total occlusion. The main cause is atherosclerotic plaque occlusion. At present, among people that are demonstrated, by coronary arteriography, to suffer from coronary heart disease, the CTO incidence is about 33%, which is increased along with the age, wherein percutaneous coronary intervention (PCI) is suitable for about 46% of the patients. The NHLBI Registry research results show that the right coronary artery occlusion is more common, then comes the left anterior descending coronary artery. All the CTO lesions develop from the earliest acute event. Due to the rupture of atherosclerotic plaques in the acute stage, thrombi lead to occluded vascular lumina; and as time lapses, the plaques rich in cholesterol lipid are gradually replaced by collagen, with which the organized thrombus structure is mixed into a fibrous structure, and in some lesions, calcification even occurs, thereby gradually developing a fiber calcification occlusion structure with loose and dense connective tissues.

Cardiac interventional therapy is one of the direct and effective approaches for CTO, but compared with non-chronic total occlusions, the operation success rate of the CTO lesions is relatively lower. In the middle of 1980s, the operation success rate was only 48%-76%, but with the improvement of the experience of the operators, the development of interventional instruments and the emergence of new interventional therapies in recent years, the operation success rate of the chronic total occlusions has been increased to 65%-92% since the late 1990s, which, however, is still far lower than that of the non-CTO lesions.

The instrument selection is a common cause for the failure of the CTO interventional therapy, and among the failures caused by instrument selection, 85% result from the failure of a guide wire in passing through the occluded lesion, followed by the failure of a balloon in passing through the occluded lesion (about 10%) or in dilation (about 5%).

Intravascular ultrasound (IVUS) is new means of diagnosis which has been applied to clinical diagnosis of vascular lesions since the 1990s, and the instrument thereof comprises two parts: a catheter with a micro-probe, and an ultrasonic imaging device. IVUS detects, using the ultrasound principle, the structure of the tissues inside the blood vessels, and the tissues of the vascular walls and surrounding the blood vessels, and is an invasive tomography technology guiding disease diagnosis and treatment. As an intravascular iconographic detection technology, IVUS increasingly demonstrates its superiority in the field of percutaneous coronary intervention, which allows for 360° real-time observation of the vascular walls from the inside, and has a resolution of 100 μm, with a projection depth of 4 mm-8 mm and a scanning range of 10 mm-15 mm. Since IVUS makes use of the phenomenon of reflection of the sound waves, it is beneficial to the display of the deep structure. In percutaneous coronary intervention, IVUS can accurately reflect the nature, the order of severity and the accumulation range of coronary artery lesions, and the diameter of the referenced blood vessels, and can therefore guide the operator to select correct strategies to treat the lesions, and help the operator to select a stent with a proper size. Moreover, IVUS can be used for evaluating the effect of coronary stent operation, which helps the operator to find and solve the problems in time after the stent has been implanted, so as to achieve the optimal effects of interventional therapy. Thus, compared with coronary interventional treatment under the guidance of coronary arteriography, the IVUS technology can further optimize the effects of coronary interventional treatment. By applying IVUS to intervention operations for coronary CTO lesions, the operation success rate and the overall technical level of the CTO intervention can be improved. In the mid-period of 1980s-1990s, the intervention operation success rate was 48%-76% in the world, and the success rate of the CTO-lesion intervention operations has been increased to 65%-92% since the late 1990s; and in China, for the experts, the intervention operation success rate at present is 70%-90%.

Due to the complexity of the structure of heart, among the existing CTO cases, very few collateral vessels meet the IVUS catheter application conditions, and the IVUS catheter cannot achieve guiding in real time the working guide wire to travel in an occlusion of the blood vessels. Moreover, due to the long learning curve and high technical requirement on the operator, the effects of using IVUS for guiding are limited, and the operation success rate is poor.

In view of the above, the present disclosure is proposed.

### DISCLOSURE OF THE INVENTION

An object of the present disclosure is to provide a coronary vein guiding system in order to solve the above problems.

In order to achieve the above object of the present disclosure, the following technical solution is employed:

the present disclosure relates to a coronary vein guiding system, comprising a coronary vein guiding catheter, and a flexible inner tube sheathed into the coronary vein guiding catheter;

the coronary vein guiding catheter sequentially comprising a coaxial section, an anti-bending section and a torsion control section;

an end portion of the coaxial section being provided with a flexible head end;

the coaxial section being connected with the anti-bending section at an obtuse angle of 136°-140°;

the anti-bending section being connected with the torsion control section at an obtuse angle of 138°-142°;

the coaxial section and the torsion control section being situated on a same side of the anti-bending section, and the coaxial section, the anti-bending section and the torsion control section being located on a same plane;

the flexible inner tube being 18 cm-22 cm longer than the coronary vein guiding catheter; and

a head end of the flexible inner tube being in the same direction as the coaxial section, and a tail end of the flexible inner tube being provided with a screw joint.

The coronary vein guiding catheter provided by the present disclosure is provided with two bending angles according to the anatomical features of heart so as to facilitate the insertion into the coronary sinus, wherein the first angle of 137°-139° helps the coronary vein guiding catheter enter the coronary vein, and the second angle of 139°-141° enables the coronary vein guiding catheter to be fixed in the coronary sinus and less prone to dislocation;

only the flexible head end of the coronary vein guiding catheter enters the coronary sinus, the flexible inner tube can freely pass through the coronary vein guiding catheter, with its head end extending from the flexible head end into the venous sinus, so as to establish a safe channel in the great cardiac vein to ensure the operation safety.

After a transfer track is established by placing the coronary vein guiding catheter into the coronary sinus, an ultrasound catheter having ultrasound or other scanning imaging functions is conveyed to the great cardiac vein, and is located at occlusions in the anterior descending branch or the circumflex branch; by collecting information and uploading the same to a workstation, a three-dimensional image of the CTO lesion is reconstructed to guide the intracoronary guide wire to travel in a true lumen in real time, within the CTO occlusion; and the catheter ultrasound is conveyed in the flexible inner tube to the designated site, which, on the one hand, can protect the probe of the catheter ultrasound, and on the other hand, can prevent the ultrasound catheter from scratching heart tissues to cause serious adverse consequences such as pericardial effusion;

in addition, the coronary vein guiding system provided by the present disclosure can further be used for establishing a drug delivery channel so as to inject drugs into heart, or draw blood through the channel established by the coronary vein guiding system;

when the ultrasound catheter and the blood drawing or drug delivery device reach the designated position, the inner tube can be withdrawn.

Preferably, in the coronary vein guiding system described above, the head end of the flexible inner tube is a blunt head end;

preferably, the flexible inner tube has a two-layered tube wall, with an inner layer being a polyvinyl pyrrolidone (PVP) hydrophilic coating and an outer layer being a high-density polyethylene (HDPE) coating;

preferably, the screw joint is made of a PC material.

Preferably, in the coronary vein guiding system described above, the coronary vein guiding catheter comprises, from inside to outside, a smooth surface layer, an elastic steel wire layer and a flexible outer layer in sequence;

preferably, the flexible head end comprises only the flexible outer layer.

Preferably, in the coronary vein guiding system described above, the smooth surface layer is a nylon polytetrafluoroethylene coating.

Preferably, in the coronary vein guiding system described above, the elastic steel wire layer is composed of meshy woven steel wires;

preferably, the elastic steel wire layer is composed of 12-16 layers of steel wires;

preferably, the steel wire is a 304 stainless steel wire.

Preferably, in the coronary vein guiding system described above, the flexible outer layer is made of a material comprising one or more selected from the group consisting of nylon, polyethylene, silica gel, polymethyl methacrylate, polyvinyl chloride, polytetrafluoroethylene, polyurethane, polyester polymers and silicone;

preferably, the flexible outer layer is made of a nylon elastomer PEBAX.

Preferably, in the coronary vein guiding system described above, the inner diameter of the coronary vein guiding catheter is 1.5 mm-1.9 mm, and the thickness of the catheter wall is 0.15 mm-0.35 mm;

preferably, the inner diameter of the flexible inner tube is 1.25 mm-1.4 mm, and the thickness of the tube wall of the flexible inner tube is 0.1 mm-0.2 mm.

Preferably, in the coronary vein guiding system described above, the length of the coaxial section is smaller than the inner diameter of the right atrium of the inserted heart.

Preferably, in the coronary vein guiding system described above, the length of the anti-bending section is smaller than the distance from the right atrium of the inserted heart to the coronary sinus; more preferably, the length of the anti-bending section is 70 mm-80 mm.

Preferably, in the coronary vein guiding system described above, the length of the flexible head end is 8 mm-12 mm.

Compared with the prior art, the advantageous effects of the present disclosure are:

having a simple structure and being adaptive to the anatomical structure of heart, being capable of greatly reducing the implementation difficulty of the heart CTO intervention operation; and being able to be used for drug delivery to heart or blood drawing.

### BRIEF DESCRIPTION OF DRAWINGS

In order to more clearly illustrate the technical solutions of the embodiments of the present disclosure or in the prior art, brief description is made below on the drawings required to be used in the description of the embodiments or the prior art. Obviously, the drawings described below illustrate only some of the embodiments of the present disclosure, and for a person of ordinary skills in the art, other drawings may be obtained from these drawings without inventive effort.

FIG. 1 is a schematic diagram of a coronary vein guiding catheter provided by the present disclosure;

FIG. 2 is a schematic structural diagram of the catheter wall of the coronary vein guiding catheter provided by the present disclosure; and

FIG. 3 is a schematic diagram of a flexible inner tube provided by the present disclosure.

### REFERENCE SIGNS

coaxial section-1;

flexible head end-101,

anti-bending section-2;

torsion control section-3;

smooth surface layer-401; elastic steel wire layer-402; flexible outer layer-403;

screw joint-501; blunt head end-502.

### DETAILED DESCRIPTION OF EMBODIMENTS

The technical solutions of the present disclosure will be clearly and completely described below in connection with the embodiments with reference to the drawings. However, it would be understood by a person skilled in the art that the described embodiments below are merely some of the embodiments of the present disclosure, rather than all the embodiments of the present disclosure, and are only used to explain the present disclosure, rather than limit the scope of the present disclosure. Based on the embodiments of the present disclosure, all the other embodiments obtained by a person of ordinary skills in the art without inventive efforts shall be covered by the scope of protection of the present disclosure. For the embodiments in which no specific conditions are given, the conventional conditions or the conditions recommended by manufacturers are used. The reagents or instruments, the manufacturers of which are not indicated, are all normal products commercially available.

In the description of the present disclosure, it is to be noted that the orientation or position relation denoted by the terms such as “center”, “upper”, “lower”, “left”, “right”, “vertical”, “horizontal”, “inner” and “outer” is based on the orientation or position relation indicated by the figures, which only serves to facilitate describing the present disclosure and simplify the description, rather than indicating or suggesting that the device or element referred to must have a particular orientation, and must be constructed and operated in a particular orientation. Therefore, said terms cannot be construed as limitations on the present disclosure. In addition, the terms “first”, “second” and “third” are only used for the description purpose, but cannot be construed as an indication or suggestion of relative importance.

In the description of the present disclosure, it should be noted that unless otherwise explicitly specified and defined, the terms “install”, “link” and “connect” shall be understood in broad sense, which may, for example, refer to fixed connection, detachable connection or integral connection; may refer to mechanical connection or electrical connection; may refer to direct connection or indirect connection by means of an intermediate medium; and may refer to communication between two elements. A person of ordinary skills in the art could understand the specific meaning of the terms in the present disclosure according to specific situations.

The present disclosure relates to a coronary vein guiding system, comprising a coronary vein guiding catheter, and a flexible inner tube sheathed into the coronary vein guiding catheter;

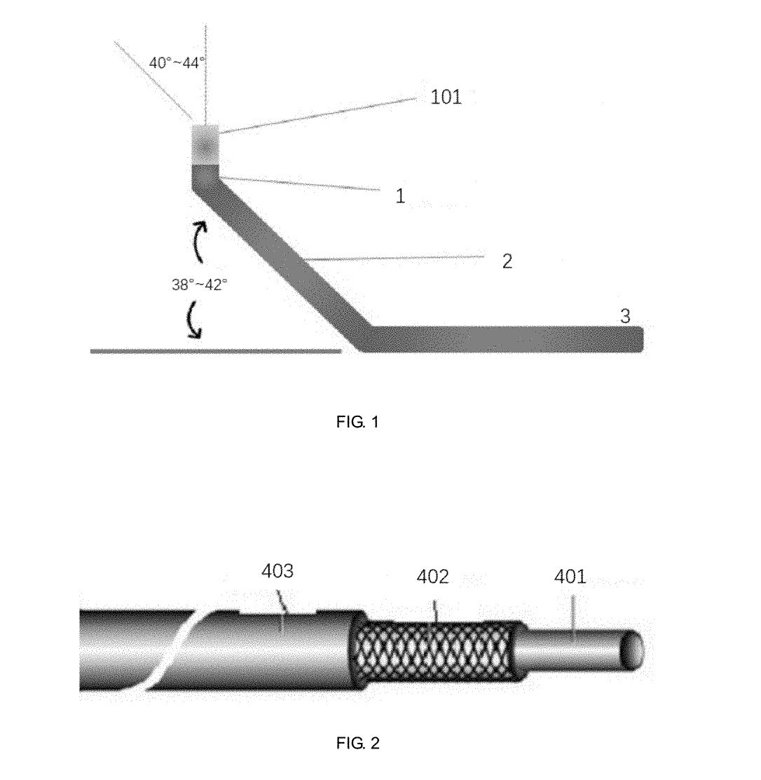

as shown in FIG. 1, the coronary vein guiding catheter sequentially comprises a coaxial section 1, an anti-bending section 2 and a torsion control section 3;

an end portion of the coaxial section 1 is provided with a flexible head end 101;

the coaxial section 1 is connected with the anti-bending section 2 at an obtuse angle of 136°-140°;

the anti-bending section 2 is connected with the torsion control section 3 at an obtuse angle of 138°-142°;

the coaxial section 1 and the torsion control section 3 are situated on a same side of the anti-bending section 2, and the three sections are located on a same plane;

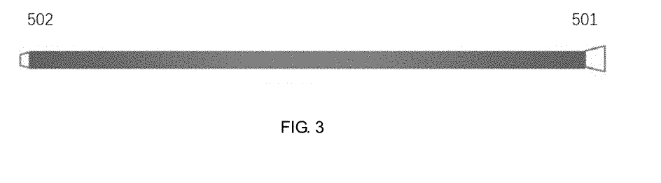

as shown in FIG. 3, the flexible inner tube is 18 cm-22 cm longer than the coronary vein guiding catheter; and

a head end of the flexible inner tube is in the same direction as the coaxial section, and a tail end of the flexible inner tube is provided with a screw joint 501.

Preferably, the head end of the flexible inner tube is a blunt head end 502;

preferably, the flexible inner tube has a two-layered tube wall, with an inner layer being a PVP hydrophilic coating and an outer layer being a HDPE high-density polyethylene;

preferably, the screw joint is made of a PC material.

The screw joint 501 can be used for the connection with an external valve, such that the interior of the flexible inner tube is a vacuum, preventing blood overflowing or air entering.

Preferably, the total length of the flexible inner tube is 125 cm-135 cm (inserted from inferior vena cava (femoral vein)) or 60 cm-70 cm (inserted from superior vena cava (subclavian vein or jugular vein));

more preferably, the total length of the flexible inner tube is 130 cm or 65 cm.

Preferably, the coaxial section 1 is connected with the anti-bending section 2 at an obtuse angle of 137°-139°, most preferably, at an obtuse angle of 138°;

preferably, the anti-bending section 2 is connected with the torsion control section 3 at an obtuse angle of 139°-141°, most preferably, at an obtuse angle of 140°.

The coronary vein guiding catheter provided by the present disclosure is provided with two bending angles according to the anatomical features of heart so as to facilitate the insertion into the coronary sinus, wherein the first angle of 137°-139° helps the coronary vein guiding catheter to enter the coronary vein, and the second angle of 139°-141° enables the coronary vein guiding catheter to be fixed in the coronary sinus without dislocation;

after a transfer track is established by placing the coronary vein guiding catheter in the coronary sinus, a guide wire having ultrasound or other scanning imaging functions is taken to the great cardiac vein, and is located at occlusions in the anterior descending branch or the circumflex branch, by collecting information and uploading the same to a workstation, a three-dimensional image of the CTO lesion is reconstructed to guide the intracoronary guide wire to travel in a true lumen in real time, within the CTO occlusion.

Preferably, in order to prevent the coronary vein guiding catheter causing injuries to the interior of heart, the bending angle is in arc transition.

The coronary vein guiding catheter enters from the subclavian vein, the jugular vein or the femoral vein, and reaches the right atrium under the guidance of the guide wire, then the torsion control section 3 is twisted such that the flexible head end 101 is inserted into the coronary sinus; then the inner tube sticks out of the coronary vein guiding catheter under the assistance of the guide wire and travels in the great cardiac vein.

The coronary vein guiding system provided by the present disclosure can be used to guide the insertion of an ultrasound catheter having ultrasound or other scanning imaging functions into the coronary sinus, and the coronary vein guiding catheter can greatly reduce the difficulty in inserting the guide wire, reduce the technical requirements on the operator, and prevent the case that the ultrasound catheter cannot pass or cause adverse consequences such as perforation.

The ultrasound catheter may preferably be selected from the IVUS catheters. The ultrasound catheter in the veins can guide, by means of imaging, the doctors to observe the position of the working guide wire for dredging the CTO occlusion segments in an intervention operation, so as to achieve the object of eliminating a CTO lesion;

in addition, the coronary vein guiding system provided by the present disclosure can further be used for establishing a drug delivery channel to inject drugs to heart; for example, injecting nitroglycerin so as to achieve the object of dilating the blood vessels;

or for drawing blood through the channel established by the coronary vein guiding system; for example, drawing blood from the coronary vein via a catheter for a blood gas analysis test to determine the lactic acid level, so as to more accurately assess the myocardial metabolism level;

when the guide wire and the blood drawing or drug delivery device reach the designated position, the inner tube can be withdrawn.

Preferably, in the coronary vein guiding system described above, as shown in FIG. 2, the coronary vein guiding catheter comprises, from inside to outside, a smooth surface layer 401, an elastic steel wire layer 402 and a flexible outer layer 403 in this order;

more preferably, the flexible head end 101 only comprises a flexible outer layer 403.

The flexible head end 101 serves to prevent scratching of the coronary sinus, while the flexible inner tube serves to incur an elastic deformation when travelling in the coronary vein guiding catheter to enable entrance in a bent manner.

In the coronary vein guiding system described above, the smooth surface layer 401 serves to facilitate the travelling of the flexible inner tube, and prevent blocking of the flexible inner tube caused by the coronary vein guiding catheter itself.

Preferably, the smooth surface layer 401 is a nylon polytetrafluoroethylene coating. Polytetrafluoroethylene is abbreviated as PTFE, and products of such material are generally referred to as “non-stick coatings”/“materials for non-stick fry pans”. PTFE has the minimum friction coefficient among plastics, and is an ideal oil-free lubrication material.

In the coronary vein guiding system described above, the elastic steel wire layer 402 serves to provide rigidity and strength for the coronary vein guiding catheter on the one hand, and provide elasticity for the coronary vein guiding catheter on the other hand, such that the coronary vein guiding catheter can travel in the veins and in the right atrium;

preferably, the elastic steel wire layer 402 can be a spring-shaped spiral structure, more preferably, the elastic steel wire layer 402 is meshy woven steel wire;

more preferably, the elastic steel wire layer 402 is composed of 12 to 16 layers of steel wires.

In the coronary vein guiding system described above, the flexible outer layer 403 serves to wrap the elastic steel wire layer 402, to prevent scratching of the myocardial tissues, and the flexible outer layer 403 shall be made of the materials having good biocompatibility and no toxicity. The flexible head end 101 is also composed of a flexible outer layer 403, but for the sake of insertion into the venous sinus and travelling of the inner tube in the great cardiac vein, the flexible head end 101 also needs to have certain strength, preferably, the flexible outer layer is made of one or more of polyethylene, silica gel, polymethyl methacrylate, polyvinyl chloride, polytetrafluoroethylene, polyurethane, polyester polymers and silicone.

Preferably, in the coronary vein guiding system described above, the inner diameter of the coronary vein guiding catheter is 1.5 mm-1.9 mm, and the thickness of the catheter wall is 0.15 mm-0.35 mm;

more preferably, the inner diameter of the coronary vein guiding catheter is 1.6 mm-1.8 mm, and the thickness of the catheter wall is 0.2 mm-0.3 mm;

more preferably, the inner diameter of the coronary vein guiding catheter is 1.7 mm, and the thickness of the catheter wall is 0.25 mm.

Preferably, the inner diameter of the flexible inner tube is 1.25 mm-1.4 mm, and the thickness of the tube wall of the flexible inner tube is 0.1 mm-0.2 mm;

more preferably, the inner diameter of the flexible inner tube is 1.3 mm-1.35 mm, and the thickness of the tube wall of the flexible inner tube is 0.15 mm.

The coronary vein guiding catheter and the flexible inner tube can be modified to have different types of inner diameters and thicknesses according to the sizes of the hearts of different patients.

Preferably, in the coronary vein guiding system described above, the length of the coaxial section 1 is smaller than the inner diameter of the right atrium of the inserted heart.

It shall be sufficient that the coaxial section 1 has a length that is smaller than the inner diameter of the right atrium, so as to be allowed to twist in the right atrium and arrive at the coronary sinus.

Preferably, in the coronary vein guiding system described above, the length of the anti-bending section 2 is smaller than the distance from the right atrium of the inserted heart to the coronary sinus; more preferably, the length of the anti-bending section 2 is 70 mm-80 mm, or can be 75 mm.

Preferably, in the coronary vein guiding system described above, the length of the flexible head end 101 is 8 mm-12 mm; or can be 9 mm-11 mm; or 10 mm.

During the use of the system, the specific position of the catheter and the inner tube can be determined by in vitro injection of a contrast agent;

preferably, in the coronary vein guiding system described above, if the method of using a contrast agent is not employed, it is also feasible to provide an indication section on the coaxial section 1 and provide a flexible inner tube indication section at the head end of the flexible inner tube, so as to indicate the position of the catheter and the inner tube;

the indication section and the flexible inner tube indication section are made of gold or platinum;

preferably, the indication section on the coaxial section is connected with the flexible head end 101.

Finally, it should be noted that the above embodiments are only used to illustrate the technical solutions of the present disclosure, rather than limit the same; although the present disclosure has been described in detail with reference to the foregoing embodiments, it should be understood by a person of ordinary skills in the art that the technical solutions described in the embodiments can still be modified, or equivalent substitution can be made to some or all of the technical features therein; and the modification or substitution would not cause the substance of the corresponding technical solutions to get out of the scope of the technical solutions of the embodiments of the present disclosure.

1. A coronary vein guiding system, comprising a coronary vein guiding catheter, and a flexible inner tube sheathed into the coronary vein guiding catheter, the coronary vein guiding catheter sequentially comprising a coaxial section, an anti-bending section and a torsion control section;
an end portion of the coaxial section being provided with a flexible head end;
the coaxial section being connected with the anti-bending section at an obtuse angle of 136°˜140°;
the anti-bending section being connected with the torsion control section at an obtuse angle of 138°˜142°;
the coaxial section and the torsion control section being situated on a same side of the anti-bending section, and the coaxial section, the anti-bending section and the torsion control section being located on a same plane;
the flexible inner tube being 18˜22 cm longer than the coronary vein guiding catheter; and
a head end of the flexible inner tube being in a same direction as the coaxial section, and a tail end of the flexible inner tube being provided with a screw joint.

2. The coronary vein guiding system according to claim 1, wherein the head end of the flexible inner tube is a blunt head end;
preferably, the flexible inner tube has a two-layered tube wall, with an inner layer being a polyvinyl pyrrolidone (PVP) hydrophilic coating and an outer layer being a high-density polyethylene (HDPE) coating;
preferably, the screw joint is made of a polycarbonate (PC) material.

3. The coronary vein guiding system according to claim 1, wherein the coronary vein guiding catheter comprises, from inside to outside, a smooth surface layer, an elastic steel wire layer and a flexible outer layer in sequence;
preferably, the flexible head end comprises only the flexible outer layer.

4. The coronary vein guiding system according to claim 3, wherein the smooth surface layer is a nylon polytetrafluoroethylene coating.

5. The coronary vein guiding system according to claim 3, wherein the elastic steel wire layer is composed of meshy woven steel wires;
preferably, the elastic steel wire layer is composed of 12˜16 layers of steel wires;
preferably, the steel wire is a 304 stainless steel wire.

6. The coronary vein guiding system according to claim 3, wherein the flexible outer layer is made of a material comprising one or more selected from the group consisting of nylon, polyethylene, silica gel, polymethyl methacrylate, polyvinyl chloride, polytetrafluoroethylene, polyurethane, polyester polymers and silicone;
preferably, the flexible outer layer is made of a nylon elastomer PEBAX.

7. The coronary vein guiding system according to claim 1, wherein an inner diameter of the coronary vein guiding catheter is 1.5˜1.9 mm, and a thickness of a catheter wall of the coronary vein guiding catheter is 0.15˜0.35 mm;
preferably, an inner diameter of the flexible inner tube is 1.25˜1.4 mm, and a thickness of a tube wall of the flexible inner tube is 0.1˜0.2 mm.

8. The coronary vein guiding system according to claim 1, wherein a length of the coaxial section is smaller than an inner diameter of a right atrium of an inserted heart.

9. The coronary vein guiding system according to claim 1, wherein a length of the anti-bending section is smaller than a distance from a right atrium of an inserted heart to a coronary sinus; preferably, the length of the anti-bending section is 70˜80 mm.

10. The coronary vein guiding system according to claim 1, wherein a length of the flexible head end is 8˜12 mm.
